(19) United States
(12) Patent Application Publication
(10) Pub. No.: US 2016/0176799 A1
(43) Pub. Date: Jun. 23, 2016

(54) ISOLATION AND PURIFICATION OF SHIKIMIC ACID

(71) Applicant: Board of Trustees of Michigan State University, East Lansing, MI (US)

(72) Inventors: Karen M. FROST, Okemos, MI (US); John W. FROST, Okemos, MI (US)

(21) Appl. No.: 14/974,656

(22) Filed: Dec. 18, 2015

Related U.S. Application Data
(60) Provisional application No. 62/094,224, filed on Dec. 19, 2014.

(51) Int. Cl. C07C 51/48; C07C 51/377; C07C 51/43; C12P 7/42

(52) U.S. Cl. CPC C07C 51/48; C12P 7/42; C07C 51/377; C07C 51/43; C07C 2101/16

(57) ABSTRACT

A method for isolating and purifying shikimic acid from a fermentation broth is provided. The method includes performing a liquid-liquid extraction on the fermentation broth with an alcohol solution to generate an extract, crystallizing solids from the extract, dissolving the solids in a second alcohol solution to generate a solution having shikimic acid, and filtering the solution having shikimic acid through a filter that does not contain ion exchange resins. Methods for dehydrating the shikimic acid to yield p-hydroxybenzoic acid are also provided.

### CROSS-REFERENCE TO RELATED APPLICATIONS

This application claims the benefit of U.S. Provisional Application No. 62/094,224, filed on Dec. 19, 2014. The entire disclosure of the above application is incorporated herein by reference.

### INTRODUCTION

The present technology relates to methods of purifying shikimic acid from multi-component solutions, such as fermentation broth.

Shikimic acid is an attractive synthon having a highly functionalized, six-membered carbocyclic ring and multiple asymmetric centers. Shikimic acid can be microbially synthesized from plant sugars or directly isolated from various plants where unconjugated shikimic acid accumulates in leaf tissue.

Chiral, as well as aromatic, chemicals can be synthesized from shikimic acid. For example, acid catalyzed dehydration of shikimic acid affords p-hydroxybenzoic acid (pHBA). Eykmann, J. F., Ber. Dtch. Chem. Ges. 24:1278 (1891). p-Hydroxybenzoic acid, which has an annual production of 14-21×10⁶ kg, is a key precursor used in the production of a variety of commercial products. For example, pHBA is a precursor for the production of parabens, which are antibacterial agents used as preservatives in cosmetics and toiletry products, among other products. Also, pHBA is a precursor for the production of a monomer used in the synthesis of liquid crystal polymers, used in electronic and automotive parts, flexible circuitry film, high-end barrier film, and high strength fibers. Additionally, shikimic acid is an essential chiral starting material in the synthesis of neuraminidase inhibitors, used in drugs that treat and prevent viral infection (e.g, influenza). Kim. C. U. et al., J. Am. Chem. Soc. 119:681 (1997); Rohloff, J. C. et al., J. Org. Chem. 63:4545 (1998). One such neuraminidase inhibitor is oseltamivir, which is marketed as Tamiflu® neuraminidase inhibitor by Genentech (San Francisco, Calif.), which is a subsidiary of Roche (Basel, Switzerland). Shikimic acid has also been used as the starting point for synthesis of a large combinatorial library of molecules. Tan, D. S. et al., J. Am. Chem. Soc. 120:8565 (1998).

Preparing p-hydroxybenzoic acid from renewable, non-toxic shikimic acid derived from plants or by fermentation as the starting materials affords significant advantages over conventional manufacturing methods. p-Hydroxybenzoic acid has been synthesized since 1860 by the Kolbe-Schmitt reaction of potassium phenoxide and carbon dioxide followed by acidification of the resulting potassium p-hydroxybenzoate. However, the starting material for the Kolbe-Schmitt reaction, phenol, is toxic and is obtained from non-renewable fossil fuel feedstocks. Additionally, in the final step of the Kolbe-Schmitt reaction, potassium p-hydroxybenzoate is neutralized (typically) with H₂SO₄. This step results in a mole of salt byproduct for each mole of p-hydroxybenzoic acid manufactured. Thus, management of significant waste streams containing sizable salt concentrations and contaminated by toxic phenol is an essential aspect of the Kolbe-Schmitt route to p-hydroxybenzoic acid.

Thus, by generating shikimic acid from renewable resources, environmentally friendly bio-based pHBA can be produced without the toxic materials commonly used to generate pHBA from benzene isolated from fossil-fuels. However, methods for isolating shikimic acid from microbes or plants known in the art require both cation and anion exchange resins for deionization and separation. Such ion exchange resins are expensive, result in dilution of shikimic acid, and generate considerable salt waste streams. Accordingly, there remains a need to develop novel methods for isolating and purifying shikimic acid from microbes or plants that eliminates the use of cation and anion exchange resins and salt waste streams associated with the use and recycling of these resins.

### SUMMARY

The present technology provides methods for isolating shikimic acid from a multi-component solution, such as a fermentation broth. Such methods include performing a liquid-liquid extraction on the solution using a suitable alcohol, such as n-butanol. The shikimic acid may be crystallized, and further purified using the alcohol.

In particular, the present technology provides methods for isolating and purifying shikimic acid from a multicomponent solution, such as a fermentation broth produced by microbial fermentation. Such methods comprise:
(a) performing a liquid-liquid extraction on the multicomponent solution with a first alcohol solution (e.g., comprising n-butanol) to generate a first extract comprising shikimic acid;
(b) crystallizing solids from the first extract comprising shikimic acid to generate a first crystalline solid;
(c) dissolving the first crystalline solid in a second alcohol solution (e.g., comprising n-butanol) to generate a second solution comprising shikimic acid;
(d) filtering the second protein solution through a filtration column that does not comprise ion exchange resins to generate an eluate comprising shikimic acid; and
(e) crystallizing shikimic acid from the eluate.

Preferably, the methods do not include the use of ion exchange resins; therefore, no salt waste is generated. Rather, the liquid-liquid extraction is performed with the alcohol, such as n-butanol or another alcohol that has a water solubility and ability to form a binary azeotrope in water similar to n-butanol. The n-butanol (or other alcohol) may be commercially-available n-butanol, redistilled n-butanol (i.e., recycled from the process), or combinations thereof.

The filtration column is employed to remove residual impurities from the shikimic acid. The filtration column is packed with a filtering material, such as diatomaceous earth, silica gel, activated carbon, charcoal, or a mixture thereof.

The current technology also provides methods for making p-hydroxybenzoic acid. The methods include:
(a) culturing bacteria, yeast or other microbe that produces shikimic acid in a fermentation broth;
(b) performing a liquid-liquid extraction on the fermentation broth with a first alcohol solution to generate a first extract comprising shikimic acid;
(c) crystallizing solids from the first extract comprising shikimic acid to generate a first crystalline solid;
(d) dissolving the first crystalline solid in a second alcohol solution to generate a second solution comprising shikimic acid;
(e) filtering the second comprising shikimic acid solution through filtration column that does not comprise ion exchange resins to generate an eluate comprising shikimic acid;
(f) crystallizing shikimic acid from the eluate; and
(g) dehydrating the shikimic acid to produce p-hydroxybenzoic acid.
   Dehydrating the shikimic acid to produce p-hydroxybenzoic acid may comprise, for example, heating the shikimic acid in an ionic liquid and H₂SO₄ at a temperature of about 120° C., wherein the ionic liquid is selected from the group consisting of 1-butyl-3-methylimidazolium salts, 1-ethyl-3-methylimidazoalium salts, 1-butyl-2,3-dimethylimidazolium salts, 1-dodecyl-3-methylimidazolium salts, 1-butylpyridinium salts, 1-butyl-2-methylpyridiniuim salts, 1-butyl-3-methylpyridinium salts, 1-butyl-4-methylpyridinium salts, 1-butyl-1-methylpyrrolidinium salts, tetra-n-pentylammonium salts, tetrabutylphosphonium salts, and combinations thereof, wherein the counter anion of the salts are bromide (Br−), chloride (Cl−), iodide (I−), hydrogen sulfate (HSO4-), tetrafluoroborate (BF4-), or hexafluorophosphate (PF6-).

### DRAWINGS

The drawings described herein are for illustrative purposes only of selected embodiments and not all possible implementations, and are not intended to limit the scope of the present disclosure.

FIG. 1 shows two reaction pathways for the generation of p-hydroxybenzoic acid; and

FIG. 2 is a flow chart that describes a method for isolating and purifying shikimic acid according to the present technology.

### DETAILED DESCRIPTION

The following description of technology is merely exemplary in nature of the composition, manufacture and use of one or more inventions, and is not intended to limit the scope, application, or uses of any specific invention claimed in this application or in such other applications as may be filed claiming priority to this application, or patents issuing therefrom. A non-limiting discussion of terms and phrases intended to aid understanding of the present technology is provided at the end of this Detailed Description.

The present technology provides methods for isolating and purifying shikimic acid from multicomponent solutions, such as from a fermentation broth. The methods include isolating and purifying shikimic acid from multicomponent solutions without the use of ion exchange resins or the salt waste streams associated with the use and recycling of the resins. Many of the solvents used in the current methods are bio-based and can be easily recycled. Additionally, the current methods do not require phase transfer catalysts for extraction of shikimic acid.

The current methods take advantage of various characteristics of shikimic acid. For example, shikimic acid is highly stable in aqueous fermentation broths, including aqueous fermentation broths heated to its boiling point. This stability allows for method steps that require elevated temperatures. Whereas other components may become denatured or negatively modified at such temperatures, shikimic acid remains intact. Shikimic acid also has good crystallinity that enables it to crystallize from solution under proper conditions, as described further below. Additionally, the high concentration of water in a binary azeotrope, such as a binary n-butanol azeotrope, and the high solubility of shikimic acid in water-saturated alcohol, such as water-saturated n-butanol, provide for extracting shikimic acid from aqueous multicomponent solutions and enables crystallization of shikimic acid upon partial concentration of shikimate-containing water-saturated n-butanol or other alcohols.

As referred to herein, a “multicomponent solution” includes any liquid mixture comprising shikimic acid or a shikimic acid salt. Such multicomponent solutions may be, for example, aqueous solutions or suspensions comprising shikimic acid and other materials. A multicomponent solution comprising shikimic acid can be generated by any means known in the art. For example, a multicomponent solution comprising shikimic acid is produced through extraction from plants. Plants and their corresponding components that are suitable for shikimic acid extraction include Agastache foeniculum (anise hyssop; leaves), Agastache rugosuin (Korean hyssop; leaves and flowers), Agastache scrophulariaefolia (purple giant hyssop), Ginkgo biloba (green leaves, spring; green leaves, autumn; and yellow leaves, autumn), Hypericum punctatum (dotted St. John's wort; leaves), Hypericum pyramidatum (great St. John's wort; leaves), Illicium anisatum (Japanese star anise; leaves), Illicium floridanum (Florida anise; leaves), Illicium henryi (leaves), Illicium lanceolatum (leaves), Illicium parviflorum (yellow anise, leaves), Illicium religiosum (leaves), Illicium simonsii (leaves), Illicium verum (Chinese star anise, fruit), Metasequoia gllyptostroboides (dawn redwood; needles), and Ribes aureum (leaves).

Shikimic acid can also be produced from genetically modified microbes, such as bacteria and fungi, such as yeasts. For example, in some embodiments, shikimic acid is produced from a carbon source in a bacteria or fungi, including yeast, genetically manipulated to comprise one or more enzyme-encoding recombinant DNA molecules, wherein the encoded enzyme is selected from the group consisting of 3-deoxy-D-arabino-heptulosonic acid 7-phosphate synthase, 3-dehydroquinate synthase, 3-dehydroquinate dehydratase, and shikimate dehydrogenase, and wherein the microbe may further comprise an inactivating mutation of at least one DNA molecule which encodes a shikimate kinase isozyme, and wherein the microbe further comprises an inactivating mutation in at least one DNA sequence which encodes a gene involved in the phosphoenolpyruvate carbohydrate phosphotransferase system; and culturing the microbe in aqueous fermentation broth containing the carbon source. Such a method for producing shikimic acid is described in U.S. Pat. No. 6,472,169, issued to Frost et al. on Oct. 29, 2002, which is incorporated herein by reference. However, it is understood that additional schemes for producing large amounts of shikimic acid from genetically modified microbes can be employed. Non-limiting examples of additional schemes for generating shikimic acid can be found in U.S. Pat. No. 6,613,552, issued to Frost et al. on Sep. 2, 2003, and U.S. Pat. No. 8,372,621, issued to Frost on Feb. 12, 2013, both of which are also incorporated herein by reference. When microbes are genetically engineered to produce shikimic acid, the microbes release the shikimic acid into aqueous fermentation broth, which is an example of a multicomponent solution according to the present technology.

Thus, for example, the present technology provides methods for producing shikimic acid comprising
(a) providing a fermentation broth comprising shikimic acid, e.g., by culturing a bacteria, yeast or other microbe comprising genetic materials that encode enzymes for the production of shikimic acid;
(b) clarifying the fermentation broth to remove cells and particulate matter to form a clarified fermentation broth;
(c) concentrating the clarified fermentation broth by boiling to form concentrated fermentation broth;
(d) acidifying the concentrated fermentation broth to form acidic fermentation broth; (e) performing a liquid-liquid extraction on the acid fermentation broth to generate a shikimic acid extract;
(f) processing the shikimic acid extract to generate a shikimic acid solution; and
(g) filtering the shikimic acid through a filtration column;
   wherein the method does not comprise the use of ion exchange resins or salt solutions required to clean and reequilibrate such ion exchange resins for reuse. Methods include those where the microbe comprises recombinant DNA molecules encoding one or more of 3-deoxy-D-arabino-heptulosonic acid 7-phosphate synthase, 3-dehydroquinate synthase, 3-dehydroquinate dehydratase, and shikimate dehydrogenase. The microbe preferably further comprises an inactivating mutation in a gene that encodes a shikimate kinase isoenzyme, or in a gene involved in the phosphoenolpyruvate carbohydrate phosphotransferase system.

### Shikimic Acid Purification

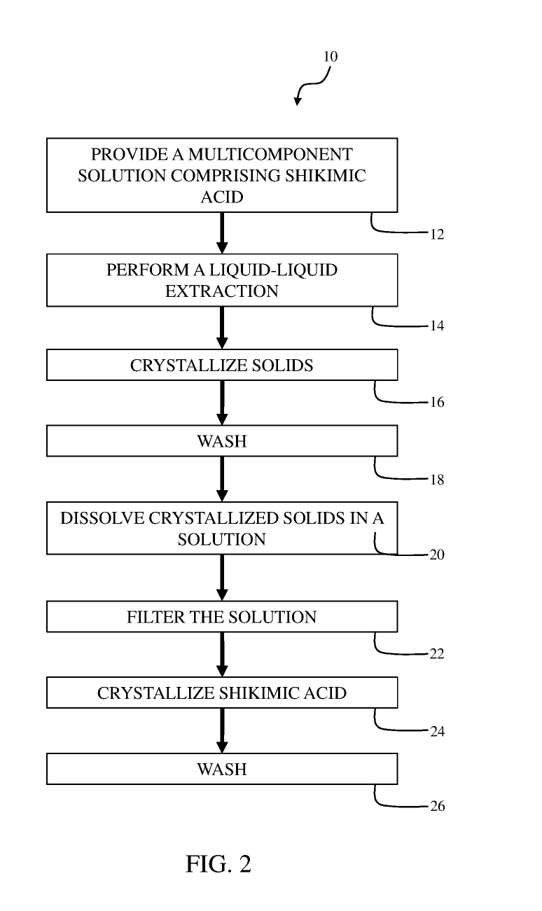

A method for isolating shikimic acid from a multicomponent solution is shown in FIG. 2. As shown in block 12, the method 10 comprises providing a multicomponent solution comprising shikimic acid. In some embodiments, the multicomponent solution is an aqueous solution derived from plants. In other embodiments, the multicomponent solution comprising shikimic acid is an aqueous fermentation broth collected from a microbial culture engineered to produce shikimic acid as described above. The scale of the microbial culture can be in the range of from about 0.5 L to about 5000 L, from about 30 L to about 3000 L, or from about 5000 L to about 50,000 L. In various embodiments, providing a multicomponent solution comprising shikimic acid includes clarifying crude fermentation broth by removing cells, cellular debris, other solids, and proteins. Clarifying can be performed by centrifuging the fermentation broth to isolate solid matter, by filtering the fermentation broth or by both centrifuging and filtering. In one embodiment, clarifying comprises two filtrations or two sequential crossflow filtrations. The first filtration may include passing crude fermentation broth through a 50-150 kD filtration cassette to remove cells, thus generating a cell-free fermentation broth. Preferably, the crude fermentation broth is passed through a 100 kD filtration cassette. The second filtration may include passing the cell-free fermentation broth through a 2-20 kD filtration cassette in order to remove protein form the cell-free fermentation broth. Preferably, the cell-free fermentation broth is passed through a 10 kD filtration cassette.

In some embodiments, the fermentation broth is processed prior to forthcoming extractions. Such processing may include reducing the volume of the broth by boiling the broth at atmospheric pressure. For example, the broth can be boiled until the volume of the broth is reduced by greater than or equal to about 50% or greater than or equal to about 75%. Processing also includes adding acid, such as for example, concentrated H₂SO₄, to the broth to reduce the pH. The pH is reduced to about pH 6, to about pH 5, to about pH 4, to about pH 3, or to about pH 2. Preferably, the pH is reduced to about 2.5. Then, the deionized water is added to the broth to increase the volume by about 15%, by about 20%, or by about 25%. After processing, the broth is a viscous black solution.

As shown in block 14, the method 10 also comprises performing at least one liquid-liquid extraction on the fermentation broth or processed fermentation broth with a first alcohol solution to generate at least one extract comprising shikimic acid. Extracting is performed by mixing the fermentation broth or processed fermentation broth with an alcohol for from about 1 hour to about 10 hours. Any alcohol in which water solubility is comparable to water solubility in n-butanol can be used for the at least one extraction. In various embodiments, the solubility is from about 50 g/L to about 80 g/L at about 25° C., preferably from about 70 g/L, to about 75 g/L. In various embodiments in which large volumes of fermentation broth are provided, the liquid-liquid extraction is performed as a counter current extraction with any alcohol provided herein.

Suitable alcohols may also include those forming a binary azeotrope in water comparable to the binary azeotrope of n-butanol in water, i.e. comprising about 55% n-butanol. Such comparable binary azeotropes include those comprising alcohols forming a binary alcohol/water azeotrope having an alcohol concentration of from about 40% to about 90%, preferably from about 50% or higher, or about 80% or lower. In some embodiments, the alcohol comprises an alcohol forming a binary alcohol/water azeotrope having a boiling point within 15° C. of the boiling point of a binary water-n-butanol azeotrope, i.e. about 92° C.

In various embodiments, the alcohol comprises a C₄-C₆ alcohol, or structural isomers thereof. For example, alcohols may be selected from the group consisting of n-butanol, structural isomers of n-butanol (e.g., sec-butanol, iso-butanol, tert-butanol), n-pentanol, structural isomers of n-pentanol (e.g., isopentyl alcohol), n-hexanol, structural isomers of n-hexanol (e.g., 2-hexanol) and mixtures thereof. In some embodiments, the alcohol comprises, or consists essentially of, n-butanol.

Unlike purification methods that require expensive ion exchange resins and salt solutions that generate a substantial amount of waste, the alcohols used herein can easily be recycled by a simple redistillation. Additionally, the alcohol can be purely (100%) commercial alcohol, a mixture of commercial alcohol and recycled alcohol, or purely (100%) recycled alcohol. In various preferred embodiments, the alcohol, or fraction thereof, is bio-based, i.e., generated from a microbial culture from a renewable feedstock. A non-limiting example of such an alcohol is bio-butanol. The liquid-liquid extraction can be performed a plurality of times in order to extract as much shikimic acid from the fermentation broth or processed fermentation broth as possible.

After the at least one extraction, the extracts having the highest concentration of shikimic acid can be processed in parallel or combined. The shikimic acid concentration can be determined by any method known in the art, such as, for example, by high performance liquid chromatography (HPLC). However, in some embodiments the extracts having the highest concentration of shikimic acid are pooled prior to continuing with the method 10. Extracts containing lower concentrations of shikimic acid can undergo an optional secondary purification described below.

In some embodiments, the extracts having the highest concentration of shikimic acid are filtered immediately upon cooling to below about 92° C. to remove particulate matter and to generate a first filtrate. For example, upon cooling to about 92° C., the extract can be suction filtered through a filter paper with a pore size of from about 1 μm to about 20 μm. In various embodiments, the pore size is about 11 p.m. The first filtrate, i.e., mother liquor, can be saved for the optional secondary purification as described below.

As shown in block 16, the method 10 then comprises forming (e.g., by crystallizing or precipitating) solids from the extracts to generate a first solids product (e.g., crystallized solids). Crystallizing is performed by reducing the volume of the extracts by evaporation until the solids begin to crystallize from the extracts. Evaporation can be facilitated by a rotary evaporator (“rotovap”). After solids begin to crystallize, the extracts are incubated at room temperature for from about 5 to about 48 hours to allow for continuing crystallization. After crystallization, the first crystalline solids are collected on sintered glass funnels with a coarse frit or similar apparatuses to generate a filter cake.

As shown in block 18, the method 10 may further comprise washing the first crystalline solids using a second alcohol. Washing is performed 1-5 times by passing the alcohol (cold) through the filter cake contained in the sintered glass funnels. Depending on the scale of the purification, each wash can be performed with from about 50 mL to about 1000 mL of alcohol or more. The second alcohol can be any alcohol described above, including the same alcohol that was used for extracting. After washing with cold alcohol, the washes are collected as a second filtrate for the optional secondary purification described below.

After washing with cold alcohol, the first crystalline solids may also be washed at least one time with from about 50 mL to about 1000 mL of cold acetone, or more depending on the scale of the purification. The cold acetone has a temperature of from about 0° C. to about −20° C. In some embodiments, the first crystalline solids are then transferred to a receptacle, such as a beaker or canister, and suspended in cold acetone for from about 1 minute to about 10 minutes with stirring. The first crystalline solids are then collected on a sintered glass funnels, or similar apparatuses, and washed 1-5 times with cold acetone. In embodiments where a plurality of extracts having high shikimic acid concentrations were purified in parallel up to this point, the first crystalline solids can now be combined.

As shown in block 20, the method 10 then comprises dissolving the first crystalline solids in a solution to generate a solution comprising shikimic acid. In various embodiments, the solution is water, such as hot water with a temperature of from about 50° C. to about 99° C. The volume of water added is at least the volume of hot water required to dissolve all of the crystalline solids. After the crystalline solids are dissolved in the water, refluxing alcohol is added to the water to increase the volume by from about 5 fold to about 15 fold. Any alcohol described above can be used, such as, for example, n-butanol.

As shown in block 22, the method 10 further comprises filtering impurities out of the solution comprising shikimic acid by passing the solution comprising shikimic acid through a filtration column or decolorizing column, to generate a third filtrate. The filtration column or decolorizing column does not contain ion exchange resins. Rather, the column is packed with a filter material selected from the group consisting of diatomaceous earth, silica gel, activated carbon, charcoal, and mixtures thereof. In various embodiments, the filtration column comprises a layer of diatomaceous earth, a later of silica gel, and a layer comprising activated carbon dispersed in silica gel. The filtration column is pre-eluted, i.e., washed, with at least 0.75 column volumes of alcohol, preferably the same alcohol that in which the shikimic acid is dissolved, and the hot solution comprising shikimic acid is loaded in the column. The liquor that passes through the column can be saved for an optional secondary purification described below. A volume of alcohol, preferably the same alcohol in which the shikimic acid is dissolved, is then added to the column, and shikimic acid is eluted. In some embodiments, elution is facilitated with pressure provided by a gas, such as nitrogen, argon, or other suitable gas. Fractions are collected, and those containing shikimic acid are pooled together to generate an eluate comprising shikimic acid. In some embodiments, the eluate is then through a sintered glass funnel with a medium frit or similar apparatus to remove additional impurities from the eluate.

As shown in block 24, the method 10 then includes crystallizing solids, including shikimic acid, from the eluate to generate second crystalline solids. Crystallizing is performed by reducing the volume of the eluate by evaporation until the solids begin to crystallize from the eluate. Evaporation can be facilitated by a rotovap. After solids begin to crystallize, the eluate is incubated at room temperature for from about 5 to about 24 hours to allow for continuing crystallization. After crystallization, the second crystalline solids are collected on sintered glass funnels with a coarse frit or similar apparatus to generate a second filter cake.

After the second crystalline solids have been collected, the method 10 comprises washing the shikimic acid, as shown in block 26. Washing is performed 1-5 times by passing cold alcohol through the second filter cake contained in the sintered glass funnels. Depending on the scale of the crystallization, each wash can be performed with from about 50 mL to about 1000 mL of alcohol or more. The alcohol can be any alcohol described above, including the same alcohol that was used for extracting. After washing with cold alcohol, the washes are collected for the optional secondary purification described below.

After washing with cold alcohol, the second crystalline solids are washed at least one time with from about 50 mL to about 1000 mL of cold acetone, or more depending on the scale of the purification. The cold acetone has a temperature of from about 0° C. to about −20° C. In some embodiments, the second crystalline solids are then transferred to a receptacle, such as a beaker, and suspended in cold acetone for from about 1 minute to about 10 minutes with stirring. The second crystalline solids are then collected on a sintered glass funnel, or similar apparatuses, and washed 1-5 times with cold acetone to yield isolated and purified shikimic acid. The isolated and purified shikimic acid is then dried, such as by a vacuum, in various embodiments. In various embodiments, the method 10 yields at least about 50 g/L, at least about 55 g/L, at least about 60 g/L, at least about 65 g/L, at least about 70 g/L, or at least about 75 g shikimic acid per liter of culture with a purity of at least about 80%, at least about 85%, at least about 90%, at least about 95%, at least about 97%, at least about 98%, or at least about 99%.

As described above, various extracts, filtrates, and washes are kept for an optional secondary purification in order to increase yields. For example, the extracts containing lower concentrations of shikimic acid, the first filtrate, all the alcohol washes, and the liquor that was passed through the filtration column may all contain residual shikimic acid that can be purified. Collectively, the extracts, filtrates, and washes, or other fraction that contain shikimic acid are referred to as “secondary samples”. These secondary samples can be pooled together or processed individually. In some embodiments, the secondary purification includes reducing the volume of a secondary sample or a combination of secondary samples by, for example, rotary evaporation, and crystallizing shikimic acid as provided above. In other embodiments, a combination of the secondary samples is pooled together, the volume is reduced, solids crystallized and washed with cold alcohol and cold acetone, and dried as provided above in order to generate additional isolated and purified shikimic acid. In yet other embodiments, at least one secondary sample can be loaded onto a filtration column or decolorizing column as provided above, after having its volume reduced, and then crystallized and washed as provided above in order to generate additional isolated and purified shikimic acid.

Production of p-Hydroxybenzoic Acid from Shikimic Acid

As discussed above, shikimic acid is a starting material for the production of p-hydroxybenzoic acid (pHBA), which is a precursor to various commercially important products. Accordingly, the present technology also provides methods for making pHBA. Such methods generally comprise production of shikimic acid according to the methods of the present technology, followed by catalyzed dehydration of the shikimic acid to make pHBA. For example, methods can include:
(a) providing a fermentation broth comprising shikimic acid, e.g., by culturing a bacteria, yeast or other microbe comprising genetic materials that encode enzymes for the production of shikimic acid;
(b) performing a liquid-liquid extraction on the fermentation broth with a first alcohol solution (e.g., comprising n-butanol) to generate a first extract comprising shikimic acid;
(c) crystallizing solids from the first extract comprising shikimic acid to generate a first crystalline solid;
(d) dissolving the first crystalline solid in a second alcohol solution (e.g., comprising n-butanol) to generate a second solution comprising shikimic acid;
(e) filtering the second solution comprising shikimic acid through a filtration column that does not comprise ion exchange resins to generate an eluate comprising shikimic acid;
(f) crystallizing shikimic acid from the eluate; and
(g) dehydrating the shikimic acid to produce p-hydroxybenzoic acid.

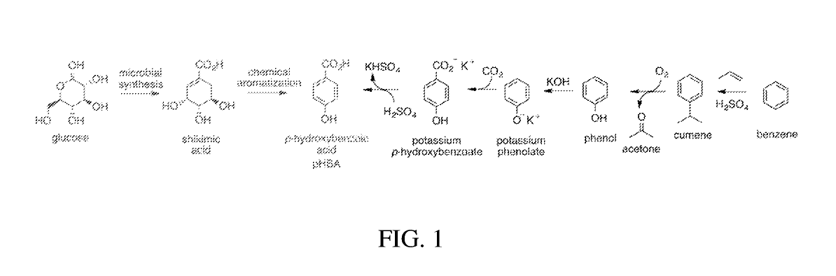

As discussed above, p-Hydroxybenzoic acid has been synthesized since 1860 by the Kolbe-Schmitt reaction of potassium phenoxide and carbon dioxide followed by acidification of the resulting potassium p-hydroxybenzoate. Reaction schemes for generating p-hydroxybenzoic acid are shown in FIG. 1. The present technology provides alternative methods for preparing p-hydroxybenzoic acid, through the use of renewable, non-toxic shikimic acid derived from plants or by fermentation as the starting materials. Such methods avoid the use of phenol derived from nonrenewable fossil fuel feedstocks and the waste streams associated with the Kolbe-Schmitt reaction.

In particular, as an alternative to the Kolbe-Schmitt reaction, p-hydroxybenzoic acid can be prepared by dehydration of shikimic acid. For example, heating shikimic acid at 120° C. with about 1 M H₂SO₄ in acetic acid solution for about 16 hours can yield about 60% p-hydroxybenzoic acid. Such a method is described in Gibson et al., Chem. Int. Ed 2011, 40, 1945-1948, which is incorporated herein by reference.

Dehydration of shikimic acid can also be accomplished with ionic liquids used as both a solvent and catalyst. For example, a suitable ionic liquid and purified shikimic acid are combined to form a reaction mixture in a container under an inert atmosphere with stirring. The container is then sealed. Non-limiting examples of suitable ionic liquids include 1-butyl-3-methylimidazolium salts, 1-ethyl-3-methylimidazoalium salts, 1-butyl-2,3-dimethylimidazolium salts, 1-dodecyl-3-methylimidazolium salts, 1-butylpyridinium salts, 1-butyl-2-methylpyridiniuim salts, 1-butyl-3-methylpyridinium salts, 1-butyl-4-methylpyridinium salts, 1-butyl-1-methylpyrrolidinium salts, tetra-n-pentylammonium salts, tetrabutylphosphonium salts, and combinations thereof, wherein the counter anion of the salts are bromide (Br−), chloride (Cl−), iodide (I−), hydrogen sulfate (HSO4-), tetrafluoroborate (BF4-), or hexafluorophosphate (PF6-). Stirring is stopped and the shikimic acid and ionic liquid are placed under active flow of an inert gas, such as N₂. The reaction mixture is then immersed in an oil bath heated to from about 100° C. to about 150° C., preferably to about 120° C., until the ionic liquid is melted and all the shikimic acid is melted in the ionic liquid to generate a reaction solution. Concentrated H₂SO₄, such as from about 5 to about 20 mol %, is added to the reaction solution and the reaction solution is incubated at room temperature with stirring under a constant flow of inert gas, such as, for example, N₂, for from about 50 to about 500 minutes, preferably from about 90 to about 150 minutes, to generate p-hydroxybenzoic acid. Optionally, the p-hydroxybenzoic acid is diluted with a mixture of acetonitrile and water, filtered, and analyzed by high performance liquid chromatography (HPLC) with a refractive index detector. In various embodiments, HPLC is performed with an Alltech® Alltima™ amino HPLC column (5 μm, 4.6 mm×250 mm) offered by Grace & Co. (Columbia, Md.) with a 60/40 acetonitrile/water buffer (90 mM ammonium formate, pH 4.0).

Although various ionic liquids can be employed as the solvent for dehydration of shikimic acid, in various embodiments the use of bromide as an ionic liquid counter anion can result in an 80% yield of p-hydroxybenzoic acid and a 20% yield of m-hydroxybenzoic acid. Such methods offer an extremely flexible strategy for synthesis of p-hydroxybenzoic acid. This follows from the opportunity to microbially synthesize shikimic acid from plant sugars or directly isolate shikimic acid from select plants where unconjugated shikimic acid accumulates in leaf tissue. All seven carbon atoms of p-hydroxybenzoic acid synthesized from shikimic acid are thus derived from CO₂. This contrasts with phenol-derived p-hydroxybenzoic acid where only one of the seven carbon atoms is derived from CO₂, using non-renewable fossil fuel resources.

Embodiments of the present technology are further illustrated through the following non-limiting examples.

### Example 1

### Isolation of Shikimic Acid from Fermentation Broth

The contents of a fermentation vessel were subjected to two sequential crossflow filtrations. In the first filtration, cells were removed from crude fermentation broth by passage through a 100 kD filtration cassette. In the second filtration, cell-free broth was passed through a 10 kD filtration cassette in order to remove protein from the broth to generate clarified fermentation broth. Clarified fermentation broth (2.82 L) containing shikimic acid (178 g) was boiled at atmospheric pressure to a volume of 700 mL, concentrated H₂SO₄ was added to pH 2.5, and the volume was readjusted to 850 mL by addition of deionized H₂O. The resulting viscous, black solution was transferred to the extraction reservoir of a liquid-liquid extractor equipped with a stir bar, and the solution was extracted sequentially for a total of 9 hours with three 1 L portions of n-butanol (3 hours per 1 L n-butanol portion). (Throughout this procedure the n-butanol was a mixture of commercial n-butanol and recycled, redistilled n-butanol, by >110° C.) The aqueous solution was stirred throughout the extraction to maximize dispersion of n-butanol in the aqueous phase.

The first two n-butanol extracts were worked up separately as follows. Hot extract was suction filtered through Whatman 1 filter paper to remove particulate, and the volume reduced by rotary evaporation until solid began to crystallize from the solution, which was then transferred to a flask and allowed to stand overnight. Crystallized solid material was collected on a sintered glass funnel (coarse frit), washed 3-4 times with 100 mL portions of cold n-butanol followed by one 100 mL portion of cold acetone (−20° C.). The crystallized solid was transferred to a beaker, stirred in 100 mL of cold acetone for 5 min., collected on a sintered glass funnel, and washed 1-2 times with 100 mL portions of cold acetone. The mother liquors from the first two extracts and the corresponding n-butanol washes were combined with the third extract and the volume was reduced to approximately one half the original volume by rotary evaporation. This afforded a second crop of shikimic acid (8.2 g, 89.9% pure).

Shikimic acid from the first extraction (85.3 g, 91.5% pure) and the second extraction (40.4 g, 95.0% pure) were combined, dissolved in 125 mL of hot water, and 1.2 L of refluxing n-butanol was added. This solution was loaded onto a 5 cm column that was packed sequentially with 10 g of Celite® 545 diatomaceous earth (available from Sigma-Aldrich Co. LLC, St. Louis, Mo.), 30 g of SiliaF1ash® P60 silica gel (available from SiliCycle Inc., Quebec City, Quebec, Canada), and a dispersion of 100 g of Darco® KB-B, 100 mesh activated charcoal (available from Sigma-Aldrich Co. LLC, St. Louis, Mo.) dispersed in 120 g of SiliaFlash® P60 silica gel. The column was pre-eluted with 1.5 column volumes of n-butanol followed by the hot aqueous solution of shikimic acid in n-butanol, and then 850 mL of n-butanol. The column was eluted under N₂ pressure. Fractions (400 mL) were collected.

Fractions containing shikimic acid were combined, passed through a sintered glass funnel (medium frit), and the volume was reduced by rotary evaporation until some crystallized solids were visible. The solution was transferred to a flask, and shikimic acid was allowed to crystallize overnight. Crystallized solids were collected on a sintered glass funnel (coarse), washed two times with 100 mL portions of cold n-butanol, and washed two times with 100 mL portions of cold acetone. The crystallized solids were transferred to a beaker, stirred in 100 ml, of cold acetone for 5 min, collected on a sintered glass funnel, and washed twice with 100 mL portions of cold acetone. Isolated crystallized material was dried by vacuum (59.4 g, 101% pure).

n-Butanol washes were combined with the mother liquors and the volume was reduced by approximately one-half by rotary evaporation. The solution was transferred to a flask and shikimic acid was allowed to crystallize overnight. Solid crystallized material was collected on a sintered glass funnel (coarse), washed two times with 100 mL portions of cold n-butanol, and washed two times with 100 mL portions of cold acetone. The crystallized solids were transferred to a beaker, stirred in 100 mL of cold acetone for 5 min, collected on a sintered glass funnel, and washed twice with 100 mL portions of cold acetone. Isolated crystallized material was dried by vacuum (38.4 g, 99.5% pure). Percent recovery of shikimic acid from the fermentation based on the first and second crop purified through the charcoal column: 54.9%, >99.5% pure.

HPLC analysis was performed on an Agilent 1100 series HPLC equipped with ChemStation acquisition software (Version XXX) from Agilent Technologies (Santa Clara, Calif.). Shikimic acid was quantified using an Alltima Amino (4.6×250 mm, 5 μm particle size) column and isocratic elution with 60/40, (v/v), CH₃CN/90 mM NH₄⁺CO₂⁻, pH 4.0 and VWD at 254 nm. A calibration curve was prepared using shikimic acid provided by Roche (Basel, Switzerland), which was pre-dried in a vacuum oven.

### Example 2

### Dehydration of Shikimic Acid in 1-Butyl-3-Methylimidazolium Bromide

1-Butyl-3-methylimidazolium bromide was dried in a vacuum oven overnight at 50° C. The dried 1-butyl-3-methylimidazolium bromide, shikimic acid, a 10 mL stripping flask, and micro stir bar were transferred to a glove bag which was purged (3×) with nitrogen. Shikimic acid (0.88 g, 5.05 mmol) and 1-butyl-3-methylimidazolium bromide (2.16 g, 9.86 mmol) were added to the 10 mL stripping flask fitted with a micro stir bar and sealed with a polyethylene stopper. The stripping flask containing the 1-butyl-3-methylimidazolium bromide, shikimic acid, and micro stir bar was removed from the glove bag and placed under an active flow of N₂ using a gas bubbler. The reaction mixture was then immersed in a 120° C. oil bath. All of the 1-butyl-3-methylimidazolium bromide melted and all of the shikimic acid dissolved in the melted ionic liquid to give a clear, yellow, viscous solution. Concentrated H₂SO₄ (0.032 mL, 0.60 mmol) was added and the reaction solution was stirred under a constant flow of N₂ for 150 min. Upon addition of the H₂SO₄, the solution darkened over the course of the reaction to yield homogenous mixtures. Aliquots (0.125 mL) were taken at t=0 min and subsequently at 30 mM timed intervals. These aliquots were added to pre-weighed 5 mL volumetric flasks. The weights of the aliquots were recorded and then diluted to 5 mL with 15/85 acetonitrile/H₂O (100 mM ammonium formate, pH 2.5). After dilution, the homogeneous mixtures were filtered with Pall 13 mm, 0.45 μM GHP membrane filters. Based on calibration curves established for shikimic acid, p-hydroxybenzoic acid, and mhydroxybenzoic acid, HPLC analysis showed formation of p-hydroxybenzoic acid (78% mol/mol) and m-hydroxybenzoic acid (19% mol/mol) at t=90 min. The HPLC column used to analyze the m-hydroxybenzoic acid and p-hydroxybenzoic acid was an Agilent Zorbax SB-C18 (5 μm, 4.6 mm×150 mm) and the buffer used was 15/85 acetonitrile/H2O (100 mM ammonium formate, pH 25). The shikimic acid was completely consumed by t=60 min. The HPLC column used to analyze the shikimic acid was a Grace Davison Alltech Alltima (5 μm amino column, 4.6 mm×250 mm) and the buffer used was 60/40 acetonitrile/H2O (90 mM ammonium formate, pH 4.0). A refractive index detector was used for all HPLC analyses as well as for establishing calibration curves.

### Non-Limiting Discussion of Terminology

The headings (such as “Introduction” and “Summary”) and sub-headings used herein are intended only for general organization of topics within the present disclosure, and are not intended to limit the disclosure of the technology or any aspect thereof. In particular, subject matter disclosed in the “Introduction” may include novel technology and may not constitute a recitation of prior art. Subject matter disclosed in the “Summary” is not an exhaustive or complete disclosure of the entire scope of the technology or any embodiments thereof. Classification or discussion of a material within a section of this specification as having a particular utility is made for convenience, and no inference should be drawn that the material must necessarily or solely function in accordance with its classification herein when it is used in any given composition.

The disclosure of all patents and patent applications cited in this disclosure are incorporated by reference herein.

The description and specific examples, while indicating embodiments of the technology, are intended for purposes of illustration only and are not intended to limit the scope of the technology. Equivalent changes, modifications and variations of specific embodiments, materials, compositions and methods may be made within the scope of the present technology, with substantially similar results. Moreover, recitation of multiple embodiments having stated features is not intended to exclude other embodiments having additional features, or other embodiments incorporating different combinations of the stated features. Specific examples are provided for illustrative purposes of how to make and use the compositions and methods of this technology and, unless explicitly stated otherwise, are not intended to be a representation that given embodiments of this technology have, or have not, been made or tested.

As used herein, the words “prefer” or “preferable” refer to embodiments of the technology that afford certain benefits, under certain circumstances. However, other embodiments may also be preferred, under the same or other circumstances. Furthermore, the recitation of one or more preferred embodiments does not imply that other embodiments are not useful, and is not intended to exclude other embodiments from the scope of the technology.

As used herein, the word “include,” and its variants, is intended to be non-limiting, such that recitation of items in a list is not to the exclusion of other like items that may also be useful in the materials, compositions, devices, and methods of this technology. Similarly, the terms “can” and “may” and their variants are intended to be non-limiting, such that recitation that an embodiment can or may comprise certain elements or features does not exclude other embodiments of the present technology that do not contain those elements or features.

Although the open-ended term “comprising,” as a synonym of non-restrictive terms such as including, containing, or having, is used herein to describe and claim embodiments of the present technology, embodiments may alternatively be described using more limiting terms such as “consisting of” or “consisting essentially of” Thus, for any given embodiment reciting materials, components or process steps, the present technology also specifically includes embodiments consisting of, or consisting essentially of, such materials, components or processes excluding additional materials, components or processes (for consisting of) and excluding additional materials, components or processes affecting the significant properties of the embodiment (for consisting essentially of), even though such additional materials, components or processes are not explicitly recited in this application. For example, recitation of a composition or process reciting elements A, B and C specifically envisions embodiments consisting of, and consisting essentially of, A, B and C, excluding an element D that may be recited in the art, even though element D is not explicitly described as being excluded herein. Further, as used herein the term “consisting essentially of” recited materials or components envisions embodiments “consisting of” the recited materials or components.

“A” and “an” as used herein indicate “at least one” of the item is present; a plurality of such items may be present, when possible. “About” when applied to values indicates that the calculation or the measurement allows some slight imprecision in the value (with some approach to exactness in the value; approximately or reasonably close to the value; nearly). If, for some reason, the imprecision provided by “about” is not otherwise understood in the art with this ordinary meaning, then “about” as used herein indicates at least variations that may arise from ordinary methods of measuring or using such parameters.

As referred to herein, ranges are, unless specified otherwise, inclusive of endpoints and include disclosure of all distinct values and further divided ranges within the entire range. Thus, for example, a range of “from A to B” or “from about A to about B” is inclusive of A and of B. Disclosure of values and ranges of values for specific parameters (such as temperatures, molecular weights, weight percentages, etc.) are not exclusive of other values and ranges of values useful herein. It is envisioned that two or more specific exemplified values for a given parameter may define endpoints for a range of values that may be claimed for the parameter. For example, if Parameter X is exemplified herein to have value A and also exemplified to have value Z, it is envisioned that Parameter X may have a range of values from about A to about Z. Similarly, it is envisioned that disclosure of two or more ranges of values for a parameter (whether such ranges are nested, overlapping or distinct) subsume all possible combination of ranges for the value that might be claimed using endpoints of the disclosed ranges. For example, if Parameter X is exemplified herein to have values in the range of 1-10, or 2-9, or 3-8, it is also envisioned that Parameter X may have other ranges of values including 1-9, 1-8, 1-3, 1-2, 2-10, 2-8, 2-3, 3-10, and 3-9.

What is claimed is:

1. A method for isolating shikimic acid from a fermentation broth or other multi-component solution, the method comprising:
a. performing a liquid-liquid extraction on the solution with a first alcohol solution to generate a first extract comprising shikimic acid;
b. crystallizing solids from the first extract comprising shikimic acid to generate a first crystalline solid;
c. dissolving the first crystalline solid in a second alcohol solution to generate a second solution comprising shikimic acid;
d. filtering the second solution through a filtration column that does not comprise ion exchange resins to generate an eluate comprising shikimic acid; and
e. crystallizing shikimic acid from the eluate.

2. The method according to claim 1, wherein the solution is a fermentation broth comprising microbially-synthesized shikimic acid.

3. The method according to claim 2, further comprising reducing the volume of the fermentation broth by greater than or equal to about 50% by boiling at atmospheric pressure.

4. The method according to claim 2, further comprising reducing the pH of the fermentation broth to about pH 2.5 prior to performing the liquid-liquid extraction.

5. The method according to claim 2, wherein crystallizing solids from the first extract comprises reducing the volume of the first extract by evaporation until crystalline solid begins to appear and then incubating at room temperature.

6. The method according to claim 2, wherein dissolving comprises adding hot water to the first crystalline solid until the first crystalline solid is dissolved and then adding refluxing alcohol to the water to increase the volume by from about 5 fold to about 15 fold.

7. The method according to claim 2, wherein the filtration column is packed with a filter material selected from the group consisting of diatomaceous earth, silica gel, activated carbon, charcoal, and mixtures thereof.

8. The method according to claim 2, wherein the filtration column is packed with a layer of diatomaceous earth, a layer of silica gel, and a layer activated charcoal dispersed in silica gel.

9. The method according to claim 2, wherein filtering comprises loading the second solution comprising shikimic acid onto the filtration column and eluting shikimic acid from the column with an alcohol.

10. The method according to claim 1, wherein the method yields shikimic acid with a purity of at least 90%.

11. The method according to claim 1, wherein at least one of the first alcohol solution and the second alcohol solution comprises an alcohol is selected from the group consisting of n-butanol, structural isomers of n-butanol, sec-butanol, iso-butanol, tert-butanol, n-pentanol, structural isomers of n-pentanol, isopentyl alcohol, sec-butanol, n-hexanol, structural isomers of n-hexanol, 2-hexanol, and mixtures thereof.

12. The method according to claim 11, wherein the alcohol comprises n-butanol.

13. The method according to claim 1, further comprising, after crystallizing shikimic acid from the eluate, converting the shikimic acid to p-hydroxybenzoic acid by contacting the shikimic acid with an ionic liquid.

14. A method for purifying shikimic acid, the method comprising:
a. culturing microbes genetically engineered to produce shikimic acid in fermentation broth;
b. collecting the fermentation broth;
c. performing a liquid-liquid extraction on the fermentation broth to generate a shikimic acid extract;
d. processing the shikimic acid extract to generate a shikimic acid solution; and
e. filtering the shikimic acid through a filtration column,
wherein the method does not comprise the use of ion exchange resins or salt solutions.

15. The method according to claim 14, wherein the liquid-liquid extraction is a counter current extraction performed with n-butanol.

16. The method according to claim 15, wherein the n-butanol is a mixture of commercial n-butanol and recycled, redistilled n-butanol.

17. The method according to claim 15, wherein the n-butanol is 100% recycled, redistilled n-butanol.

18. The method according to claim 14, wherein filtering comprises packing a column with a layer of diatomaceous earth, a layer of silica gel, and a layer comprising activated charcoal dispersed in silica gel, and washing the column with at least 1 column volume of n-butanol.

19. The method according to claim 14, wherein collecting the fermentation broth comprises clarifying crude fermentation broth by two sequential crossflow filtrations, the first filtration comprising passing the crude fermentation broth through a 100 kD filtration cassette to generate a cell-free broth, and the second filtration comprising passing the cell-free broth through a 10 kD filtration cassette to remove proteins.

20. A method for making p-hydroxybenzoic acid, the method comprising:
a. culturing bacteria, yeast or other microbe that produces shikimic acid in a fermentation broth;
b. performing a liquid-liquid extraction on the fermentation broth with a first alcohol solution to generate a first extract comprising shikimic acid;
c. crystallizing solids from the first extract comprising shikimic acid to generate a first crystalline solid;
d. dissolving the first crystalline solid in a second alcohol solution to generate a second solution comprising shikimic acid;
e. filtering the second comprising shikimic acid solution through filtration column that does not comprise ion exchange resins to generate an eluate comprising shikimic acid;
f. crystallizing shikimic acid from the eluate; and
g. dehydrating the shikimic acid to produce p-hydroxybenzoic acid.

21. The method according to claim 20, wherein dehydrating the shikimic acid to produce p-hydroxybenzoic acid comprises heating the shikimic acid in an ionic liquid and H₂SO₄ at a temperature of about 120° C.

22. The method according to claim 21, wherein the ionic liquid is selected from the group consisting of 1-butyl-3-methylimidazolium salts, 1-ethyl-3-methylimidazoalium salts, 1-butyl-2,3-dimethylimidazolium salts, 1-dodecyl-3-methylimidazolium salts, 1-butylpyridinium salts, 1-butyl-2-methylpyridiniuim salts, 1-butyl-3-methylpyridinium salts, 1-butyl-4-methylpyridinium salts, 1-butyl-1-methylpyrrolidinium salts, tetra-n-pentylammonium salts, tetrabutylphosphonium salts, and combinations thereof, wherein the counter anion of the salts are bromide (Br−), chloride (Cl−), iodide (I−), hydrogen sulfate (HSO4-), tetrafluoroborate (BF4-), or hexafluorophosphate (PF6-).
